(19) United States
(12) Patent Application Publication
(10) Pub. No.: US 2013/0088685 A1
(43) Pub. Date: Apr. 11, 2013

(54) Iris Cameras

(71) Applicant: Andrew Holland, Aylesbury (GB)

(72) Inventor: Andrew Holland, Aylesbury (GB)

(21) Appl. No.: 13/646,419

(22) Filed: Oct. 5, 2012

(30) Foreign Application Priority Data
Oct. 7, 2011 (GB) 1117356.4

(51) Int. Cl. A61B 3/11; A61B 3/14

(52) U.S. Cl. 351/206; 351/246

(57) ABSTRACT

An iris camera comprising a lens system, an image sensor arranged to capture images of an eye acquired by the lens system, and a processing unit, is described. The processing unit comprises an image analysis module adapted to measure pupil dilation of an image of an eye captured by the image sensor; and a control module arranged to generate a control signal to control a light source for illuminating the eye, the control module being arranged to generate the control signal to activate the light source to illuminate the eye when the measured pupil dilation is greater than a predetermined threshold.

### CROSS REFERENCE TO RELATED APPLICATIONS

This application claims the right of priority under 35 U.S.C. §119 to GB application serial no. 1117356.4, filed Oct. 7, 2011, which is incorporated by reference in its entirety.

### FIELD

This invention relates to systems, apparatus and methods for capturing iris images. The approach provides increased reliability of identity checks using iris recognition technology.

### BACKGROUND

The human iris is a muscle for controlling pupil dilation and consequently how much light enters the eye for image formation on the retina. This muscle has such rich variations in pigmentation patterns across the global population that no two people ever have the same patterns. Even twins and eyes of the same individual are greatly different and this provides an opportunity for powerful identity checking technology. It is a result of the vast number of degrees of freedom inherent in iris patterns compared with the size of the global population, or even compared with the number of humans that have ever or will ever exist, that iris recognition offers such a powerful opportunity for identity verification. Iris recognition technology is much better at differentiating between individuals than traditional methods such as presenting original photo documentation, signing, using chip and PIN and fingerprint matching.

Systems based on iris recognition are applicable in a range of applications including commercial and official contexts where identity checks are important for commercial, legal, security or other reasons. For example, certain airports currently employ the use of iris cameras for verifying the identity of individuals crossing national borders.

Iris cameras for use in iris recognition technology must obtain images that can be checked against reference images forming part of a user profile for that individual. The iris camera is therefore one element of the whole iris recognition system, where other elements include a database storing reference images, or at least data derived from such images, as part of a remote user profile for registered users of iris recognition. If the user profile contains reference data derived from an initial reference image, the reference data acts like a barcode uniquely identifying that user. This saves on memory required to store the material against which future identity checks are made.

In order to register for iris recognition, a user must have reference images of their irises taken for their user profile. An iris camera is clearly required for this registration process, as well as for subsequent instances when the technology is being used by the user to access various rights or authorize a transaction. However, the camera used in the registration step is not necessarily the same camera as the one used in subsequent instances because of course a user may register, for example, at a bank and subsequently require ATM services elsewhere gaining access by iris recognition.

Iris cameras generally have a lens system, an image sensor and a central processing unit for controlling automatic focusing systems and other functionality of the camera itself. Camera designs must take account of various practical issues related to providing a convenient, user-friendly arrangement, including for example providing a reasonably sized capture box in which a user's irises can be properly imaged. This means that a user does not have to worry about positioning his or her eyes exactly within a small distance range from the camera and at a very precise level and so on. The camera must also work in different ambient conditions and should work across the range of iris colorings across the global population.

In order to provide an iris image with enough information for reliable identification purposes, an iris image of sufficiently high resolution must be captured. However, image resolution is not the only factor determining the amount of useful information contained in the image. If the pupil is highly dilated, then the area of the iris may be too small to provide enough usable image data for identification purposes. When the amount of information in an iris image is small, the reliability of any identity checks based on that image will be low and the error rate will be increased. Furthermore, there will be a significant chance of generating a false positive, resulting in provision of rights to an unentitled or fraudulent user.

The present invention seeks to address some or all of the above issues.

### SUMMARY

According to a first aspect of the present invention there is provided an iris camera comprising a lens system, an image sensor arranged to capture images of an eye acquired by the lens system, and a processing unit, the processing unit comprising: an image analysis module adapted to measure pupil dilation of an image of an eye captured by the image sensor; and a control module arranged to generate a control signal to control a light source for illuminating the eye, the control module being arranged to generate the control signal to activate the light source to illuminate the eye when the measured pupil dilation is greater than a predetermined threshold.

The control software is further adapted to switch on the light source when the measured pupil dilation is greater than a predetermined threshold. As a result, the pupil can be constricted to increase the area of the iris. This makes it possible to capture an image of the iris having a greater amount of information. Furthermore, if pupil dilation does not react to the illumination as expected, this can advantageously lead to detection of various forms of fraudulent use, including use of iris relaxants to keep the pupil dilated, presentation of an image of an iris on printed media or presentation of digitally streamed media showing an iris.

Preferably, the iris camera re-captures and re-analyses image data once the light source illuminates the iris in order to check that the light source has constricted the pupil sufficiently for capturing a suitable image (i.e. an iris image with sufficient information content for reliable identification). The iris camera preferably outputs an iris image only if pupil dilation is at or below the predetermined threshold to ensure that all output images contain a predetermined minimum threshold amount of information. The image analysis software may measure pupil dilation by locating a pupil-iris boundary and an iris-sclera boundary by searching for areas of sudden change in contrast and calculating a measure of pupil dilation based on the locations of the boundaries with respect to the pupil centre. The measure of pupil dilation may be ratio between the pupil radius and a radial distance between the two boundaries. This provides a simple way of quantifying pupil dilation with respect to the size of the iris. This is equivalent to providing a measure of iris area with respect to the total area within the circular iris-sclera boundary—in other words a measure of the availability of the iris for imaging.

Advantageously, the control software instructs the light source, when the light source is on, to emit pulses of light since pupil constriction occurs naturally in response to increased illumination. The pulses are preferably emitted at times that are not predictable by the user. For example, at least one pulse may be emitted during a frame selected from a group of consecutive frames, the selected frame not being predictable by the user. This enables detection of fraudsters who are using printed media or digitally streamed media in an attempt to pose as a legitimate user. The pupil shown on a printed image clearly will not respond at all changes in illumination. Furthermore, if the user cannot predict when the light pulses will occur, it is impossible for all practical purposes to make the digitally streamed media shown pupil constrictions at the times that correspond to the pulses. Such a lack of correspondence can be detected as an indication of fraudulent use by the central processing unit using image analysis software and other applications.

The light source may be part of the iris camera, may be directly ahead of the iris being imaged so that the light emitted is used efficiently for pupil constriction and may be slightly below the level of the iris being imaged to avoid shadows being cast across the iris by eyelashes and eyebrows. The light source may comprise light emitting diodes (LEDs) which may emit white light.

The control software may be further adapted to generate a ‘request failed’ message if, after a predetermined duration or a predetermined number of cycles of image re-capture and re-analysis, pupil dilation is still greater than the predetermined threshold. This enables detection of the use of iris relaxants or printed media or other related types of fraudulent use.

According to a related aspect, a method of controlling the quality of iris captured images output from an iris camera is provided. The method comprises: obtaining an initial image of an iris of a user from the camera; measuring pupil dilation of the iris using image analysis techniques on the captured image; comparing the measured pupil dilation with a predetermined threshold; illuminating the iris and capturing a further image, if the determined pupil dilation is greater than the threshold; and outputting a validated iris image, if the comparing step shows that the determined pupil dilation is not greater than the predetermined threshold. This ensures that all output images have a minimum threshold information content, and enables detection of fraudulent use including by use of iris relaxants, printed media and digitally streamed media.

Pupil dilation may be measured by locating a pupil-iris boundary and an iris-sclera boundary using a protocol that searches for areas of sudden change in contrast, and calculating a measure of pupil dilation based on the locations of the boundaries with respect to the pupil centre. The measure of pupil dilation may be a ratio between the pupil radius and a radial distance between the two boundaries.

### BRIEF DESCRIPTION OF THE DRAWINGS

Specific embodiments of the invention will now be described, by way of example, with reference to the accompanying drawings, of which:

FIG. 1 is a schematic block diagram showing an iris camera according to a first embodiment of the invention, the iris camera being shown together with a server and a remote biometric matching engine;

FIG. 2 is a flow chart showing a method performed by the elements of FIG. 1;

FIG. 3A is a schematic diagram of an eye showing pupil and iris boundaries and a radio of distances used as a measure of pupil dilation;

FIG. 3B is a series of schematic diagrams of the eye showing a threshold pupil dilation together with an over-dilated pupil and a pupil that is not over-dilated;

FIGS. 4A, 4B and 4C are a series of schematic diagrams of the eye and the iris camera of FIG. 1 showing the method of image capture according to an embodiment of the invention; and

FIGS. 5A, 5B and 5C are a series of schematic diagrams of the eye and the iris camera of FIG. 1 showing a further method of image capture according to an embodiment of the invention.

### DETAILED DESCRIPTION

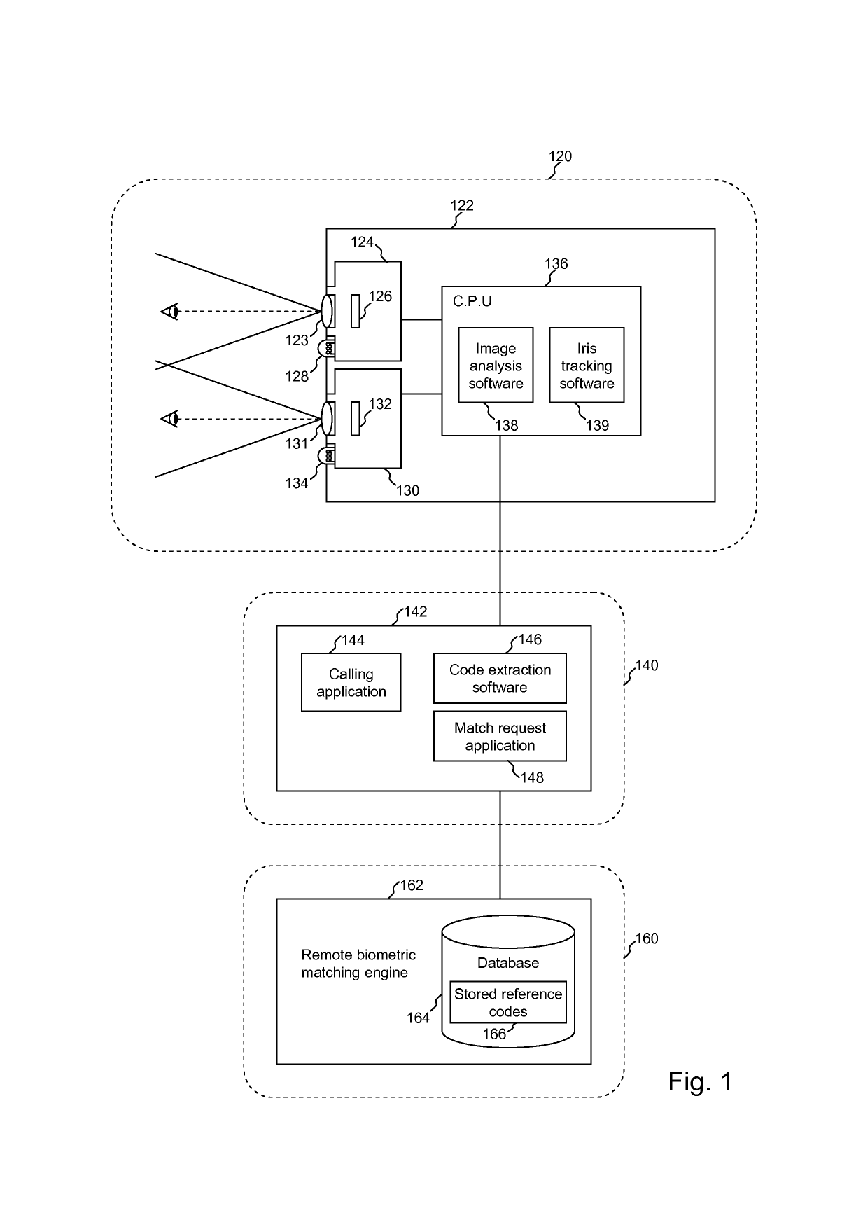

The elements of an iris camera 122 embodying the present invention are shown in FIG. 1, together with a server 142 with which, in use, the camera 122 communicates, and a remote biometric matching engine 162 in communication with the server 142. The iris camera 122 supplies quality-assured iris images to the server 142 for use in biometric identification checks that are run by checking against reference data stored in the matching engine 162.

The server 142 may be a client personal computer (PC), a client computer network or other computing infrastructure of or for the client. Communication with the server 142 may be indirect via a host of the iris camera and/or the client, including through a host switch which enables communication between a camera domain 120 and a client domain 140. The client domain 140 may communicate with a network of iris cameras through such a switch. A calling application 144 for requesting sets of iris images may be provided in the client software. Applications 146 for extracting unique codes from incoming images may also be provided here, so that data files smaller than whole images may be sent to the matching engine 162 for faster matching. In this regard, a match request application 148 is provided in the server for constructing a matching request and communicating with the matching engine 162.

The client is any organisation requiring that the identity of individuals be verified before certain permissions or access can be granted or certain transactions can be carried out. Examples include banks, point of sale (POS) terminals, auto-telling machine (ATM) terminals, high security departments or facilities and national border controls. A range of clients may be served by a single matching domain 160.

The biometric matching engine 162 comprises a computing infrastructure housing a database 164 containing files 166 relating to users for the purpose of verifying their identity in biometric security checks. The matching domain 160 may be remote from both the camera and client domains 120, 140 and may communicate with the client domain 140 either directly or via one or more hosts, including via a switch so that it may communicate with a network of clients.

Some aspects of the present embodiment relate to the elements inside the camera domain 120. In the embodiment of FIG. 1, the camera 122 comprises left and right image acquiring assemblies 124, 130, one for each iris, and a central processing unit (CPU) 136 which may, for example, be an embedded chip or a PC. In the embodiment shown, each image acquiring assembly 124, 130 has a triplet lens system 123, 131, automatic fine-focus and a 1.3 Mpix image sensor 126, 132, as well as a light source 128, 134 just below a lens aperture. In other embodiments there may for example be different lens systems and a differently positioned light source. The image sensors 126, 132 digitize images formed by the optics of the image acquiring assemblies and provide them to the CPU 136 for processing. The processing is carried out in the CPU using various applications 138, 139 which analyse the images and send instructions back to the two image acquiring assemblies 124, 130 for making adjustments. These adjustments may, for example, be performed as part of an automatic fine-focus system, or as part of iris-tracking in which adjustments are made to keep the pupil centred in any subsequently captured images.

Embodiments of apparatus and methods for detecting when a pupil is over-dilated and for attempting to constrict it will now be described with reference to FIGS. 1, 2 and 3.

A user is presented at Step 200 to the iris camera so that their left and right irises are located inside the capture boxes of the left and right image acquiring assemblies, 124 and 130, respectively. In this configuration, the camera performs protocols such as automatic fine-focus and iris tracking to prepare for iris capture and to make sure the irises remain centred and in-focus. With the irises visible and in-focus, the image sensors generate at Step 202 initial image data which is then used for checking pupil dilation.

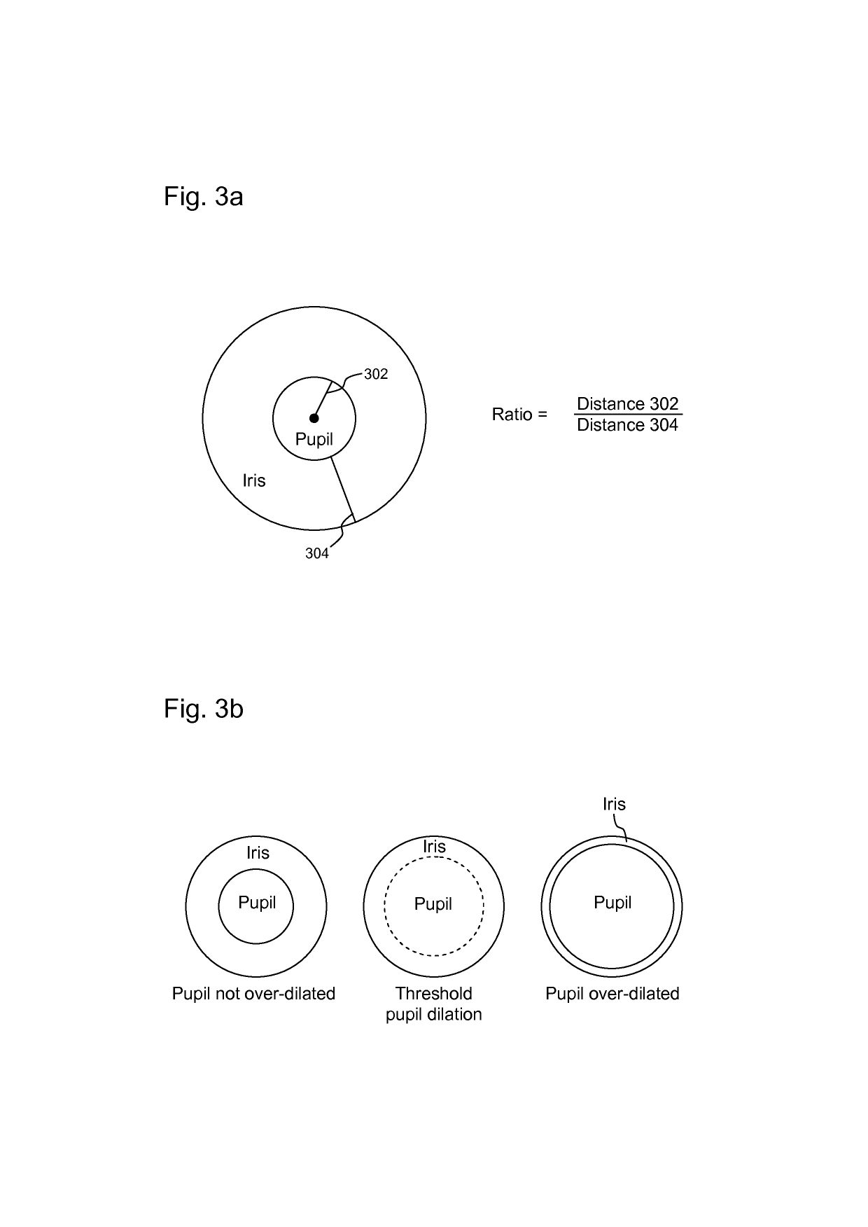

The extent to which each pupil is dilated can be measured by running at Step 204 image analysis software to identify the boundaries between the pupil and the iris, and the iris and sclera. Referring to FIG. 3A, measurements are made at Step 206 of the distance 302 from the pupil centre to the pupil/iris boundary and of the distance 304 from the pupil/iris boundary to the iris/sclera boundary. The processor (CPU) can then calculate a ratio of the two which provides a measurement of the extent of pupil dilation. In this approach, boundaries are identified by looking for areas in the captured images with sudden changes in contrast (in a similar way to methods used in iris-tracking software).

Based on the measured ratio, the image analysis software determines at Step 208 whether the pupil is over-dilated. If the measured ratio exceeds a pre-determined threshold (FIG. 3B), the pupil is over-dilated and an attempt is made to constrict it. If the ratio is at or below the threshold value, the iris area is sufficiently large for image capture and analysis.

In other embodiments, just the pupil radius could be measured. In yet further embodiments, the area of the iris could be measured. These alternative measurements all provide an indication of the extent to which the pupil is dilated.

If the pupil is over-dilated, the system attempts to correct this by constricting the pupil. An instruction signal is generated and sent at Step 210 from the CPU 136 to the image acquiring assemblies 124, 130 to switch on (activate) the light sources 128 and 134 and illuminate the eyes. These light sources comprise pulsing white LEDs positioned directly ahead of and slightly below each of the user's eyes to induce natural constriction of the pupils under conditions of increased illumination. When on, the LEDs emit several light pulses per second, these pulses being randomly distributed among the captured 30 frames per second of the resultant images.

In other embodiments, the light source provides a constant illumination of the iris when activated (as opposed to emitting light pulses). Some embodiments may use other forms of electric lighting for the light source.

After having attempted to constrict the pupil using the light source, the camera 122 generates at Step 212 image data and analyses it at Step 214 to re-measure at Step 216 pupil dilation. If the pupil is still over-dilated with respect to the threshold level (FIG. 3B), imaged data may again be re-generated, re-analysed, and pupil dilation re-measured (repeating Steps 212, 214 and 216), and if after several iterations, a timer has expired as determined at Step 220, and the pupil is still over-dilated, the LEDs are switched off at Step 222 and the CPU generates at Step 224 a ‘request failed’ message. In security control applications, this eventuality may indicate that a fraudster has tried to fool the system by taking iris relaxants to keep the pupil over-dilated. Alternatively, if use of the iris camera is not supervised by a security officer or other human operator, a ‘request failed’ message may indicate the use of printed media showing an image of an iris in an attempt to pose as someone else.

If the illumination by the LEDs has successfully constricted the pupils—or if the pupils were not over-dilated in the first place—a set of quality iris images, each having an information content of at least a predetermined minimum threshold value, is captured and provided to the server, and the LEDs (if on) can be switched off (all at Step 226).

The server 142 then extracts at Step 228 a code from the image set that uniquely identifies the user and provides this to the matching engine 162. The matching engine 162 then searches at Step 230 for matches with the stored codes 166.

If a code matching the one generated from the image set is found at Step 232, then an associated user profile can be found, thus identifying the user. A ‘positive match’ message is generated at Step 236 and the information identifying the user is sent from the matching engine 162 to the server 142. If the verified identity is the same as the identity the user is claiming, the user will be granted rights or access as appropriate. If, however, the user is posing as someone else, the system will recognise this and will not grant access. Consequently, a ‘positive match’ message is not always associated with the provision of rights.

If a matching code is not found at Step 232 in the database 164, a ‘no match’ message is generated at Step 234 and sent from the matching engine 162 to the server 142. This means that there is no profile associated with the user who is presented at the iris camera. A ‘no match’ message my indicate, for example, that a legitimate user is attempting to access their bank account at an ATM but that they have not been registered for iris recognition and consequently do not have a reference code 166 and user profile stored in the database 164. Alternatively, it may indicate that a fraudster not registered with a profile in the database 164 is attempting to access someone else's account.

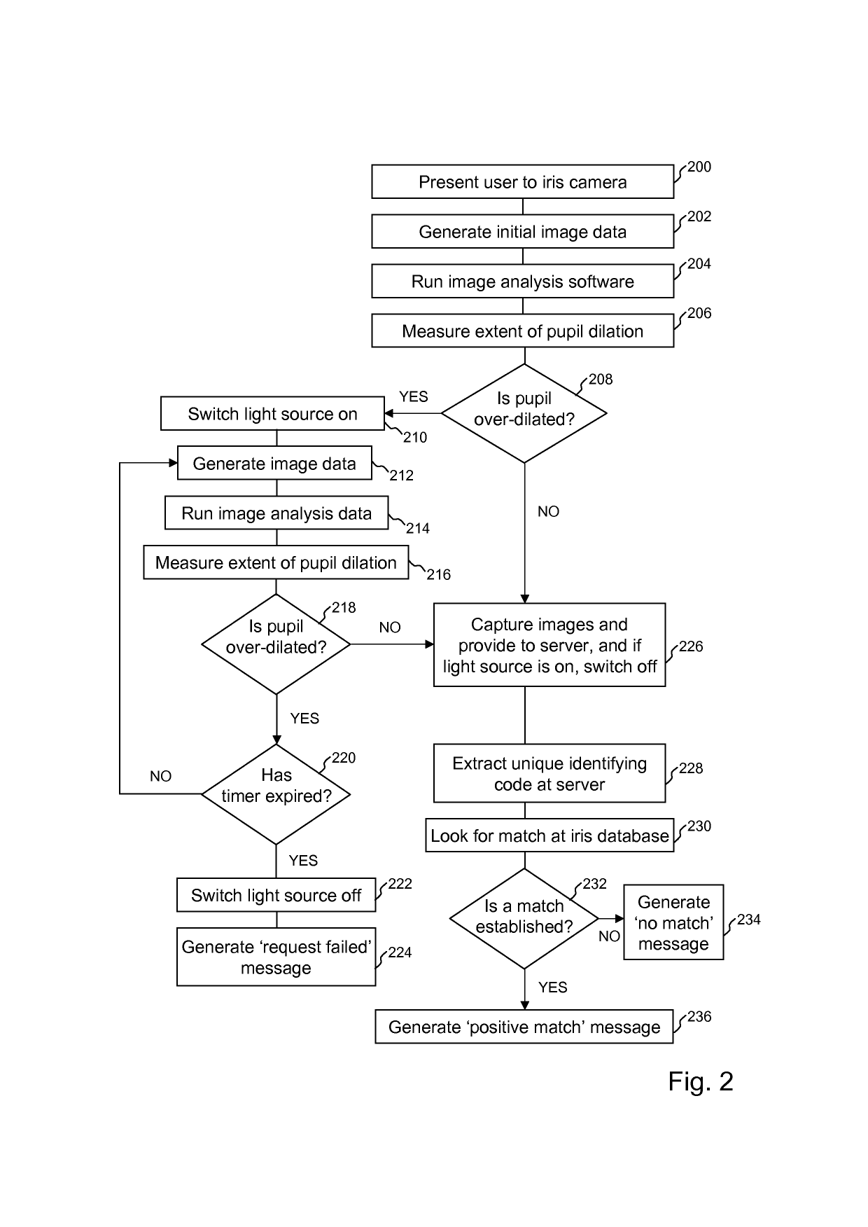

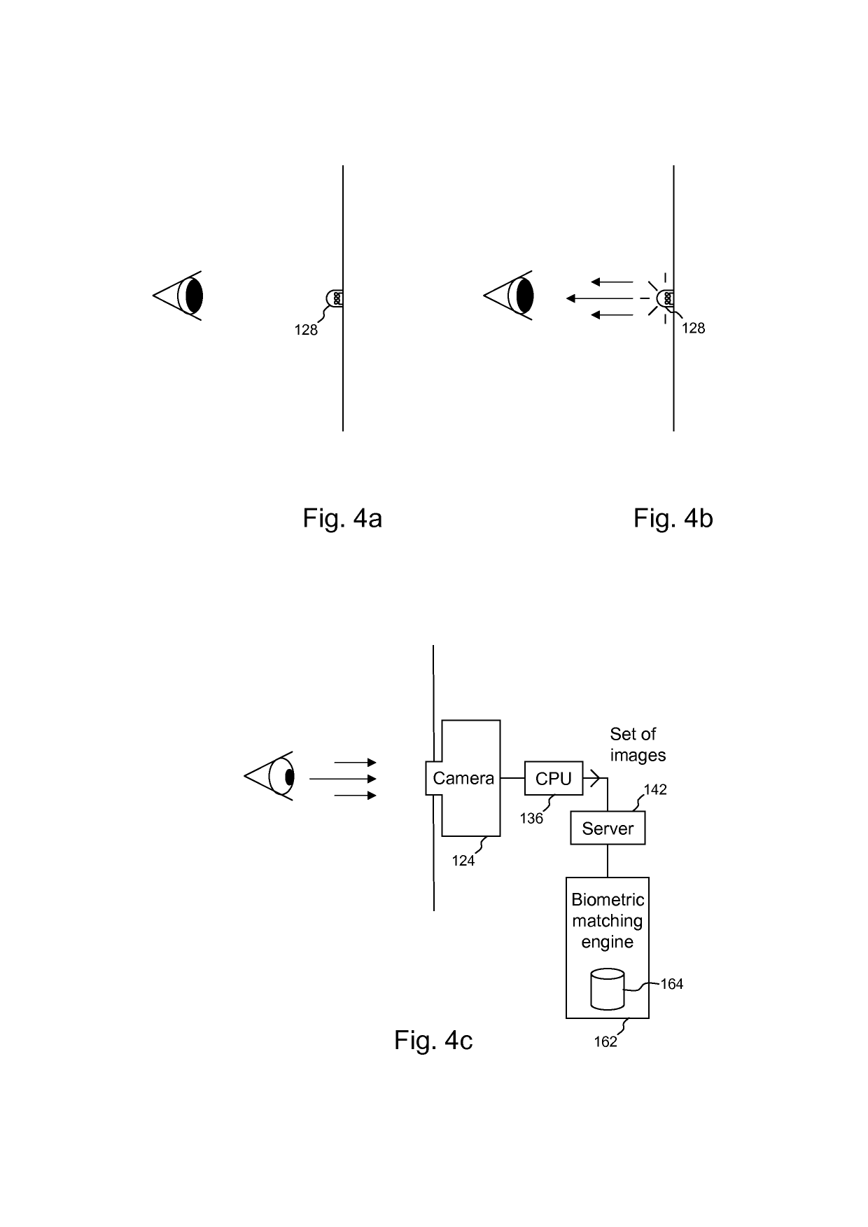

Aspects of the present embodiment are well suited to solving the problem of legitimate ATM users trying to access their bank account with naturally dilated pupils after dark. At dawn, dusk and after dark, there is a risk that a user's pupils will be too dilated for them to be successfully identified even if they are legitimate account holders registered to use iris recognition technology. An embodiment of a method of accessing a user bank account in these conditions will now be described with reference to FIGS. 2 and 4.

Initially, if an over-dilated pupil is presented to the iris camera (FIG. 4a) the camera will measure the extent of pupil dilation (step 206, FIG. 2) and will switch on at Step 210 the light sources 123, 131 to illuminate the eyes (FIG. 4b). This should constrict the user's pupils (FIG. 4c) and the camera will re-generate and re-analyse at Steps 212, 214 image data and re-measure at Step 216 the extent of pupil dilation. The iris area should now be sufficiently large for quality images to be captured for reliable user identification. In this case, the iris camera 122 captures images at Step 236 and provides them to the server 142. The legitimate, registered user is identified at Step 236 and access to his or her account is granted.

The idea behind this approach is to make access to services for legitimate users as reliable as possible, and to minimise the likelihood that a legitimate user cannot access their account. Variations or other applications of this approach include use of iris recognition to pay for shopping in everyday contexts such as at petrol stations with pay-at-pump facilities after dark.

The ATM user is preferably not disturbed by, or even aware of, any illumination required to constrict their pupils.

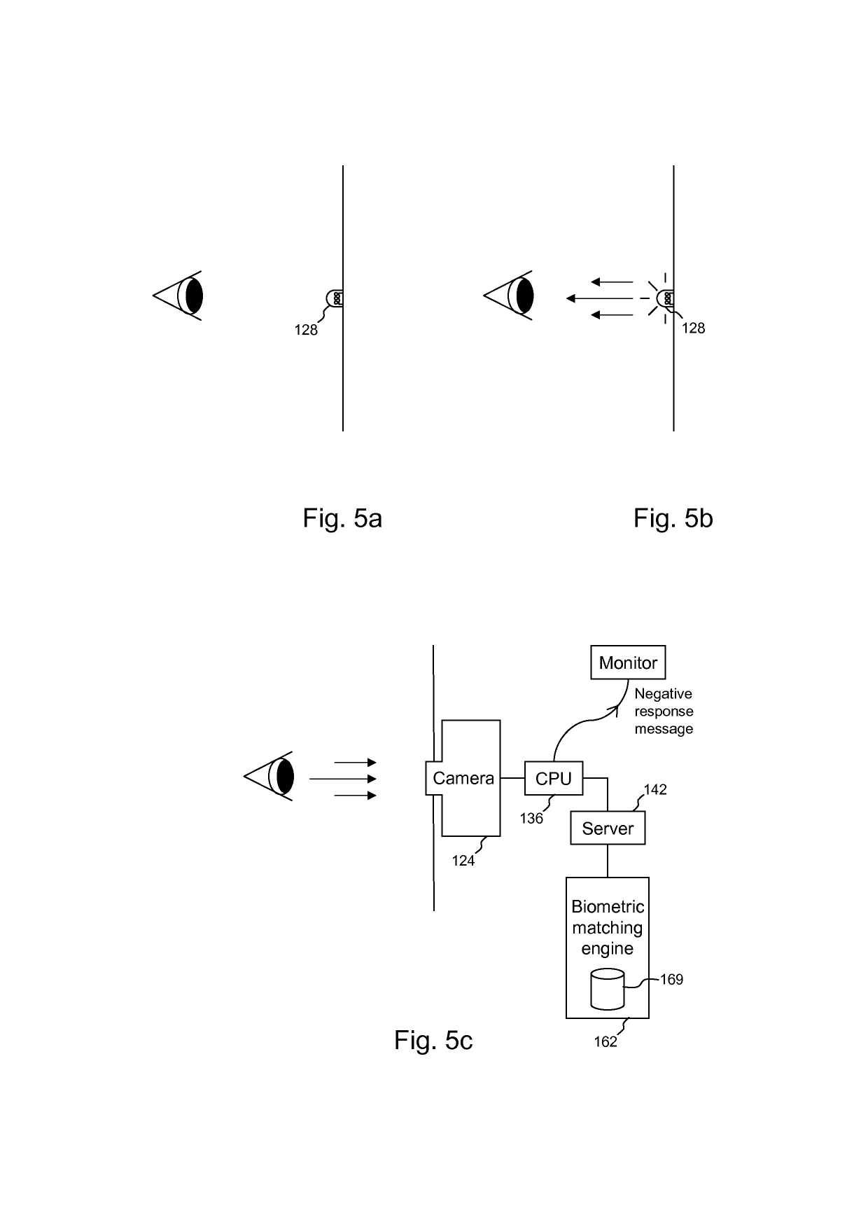

Other aspects of the described embodiments are well suited to addressing the problem of detecting fraudsters in security applications such as national boarder control in airports. An embodiment of a method according to this approach will now be described with reference to FIGS. 2 and 5.

In embodiments implemented at an airport security checkpoint, users with over-dilated pupils are detected at Step 208 (FIG. 5a). The camera 122 attempts at Step 210 to constrict the pupils (FIG. 5b) and if the pupil does not respond, this is detected at Step 216 and a security officer is alerted that the user may have taken iris relaxants (FIG. 5c). Variations on this approach include checking for iris relaxants using illumination even when pupils are not over-dilated. The threshold for an over-dilated pupil may be lower in this high-security application compared with the example discussed above of legitimate ATM use after dark.

If fraudsters present printed media showing photographs of irises in an attempt to fool an unmanned iris camera, this will also be detected because the printed media will not respond when the light sources 123, 131 are switched on at Step 210. Similarly, if digitally streamed media showing an iris is presented, it is very unlikely that the timing of any constrictions of the streamed pupil will correspond to the randomly timed illumination pulses of the LEDs.

The idea behind this approach is to maximise the likelihood of detecting fraudsters. Other applications include, for example, use in government buildings and high security prisons.

Having specifically described embodiments of the present in detail, it is to be appreciated that the above described embodiments are exemplary only and that modifications will occur to those skilled in the art without departure from the spirit and scope of the present invention. For example, even though a specific pulsed proximity sensor has been described any form of proximity sensor which give and accurate reading relatively quickly could be used.

What is claimed is:

1. An iris camera comprising a lens system, an image sensor arranged to capture images of an eye acquired by the lens system, and a processing unit, the processing unit comprising:
an image analysis module adapted to measure pupil dilation of an image of an eye captured by the image sensor; and
a control module arranged to generate a control signal to control a light source for illuminating the eye, the control module being arranged to generate the control signal to activate the light source to illuminate the eye when the measured pupil dilation is greater than a predetermined threshold.

2. An iris camera according to claim 1, wherein the image sensor, image analysis module and the control module are arranged to re-capture and re-analyse image data if the light source has been activated.

3. An iris camera according to claim 1, further arranged to output a valid captured iris image if pupil dilation of the captured image is at or below the predetermined pupil-dilation threshold.

4. An iris camera according to claim 1, wherein the image analysis module is arranged to measure pupil dilation by:
locating a pupil-iris boundary and an iris-sclera boundary in the captured image by searching for regions having a sudden change in contrast; and
calculating a measure of pupil dilation based on locations in the captured image of the boundaries with respect to a location of the pupil centre.

5. An iris camera according to claim 4, wherein the measure of pupil dilation is calculated by the image analysis module to be a ratio in the captured image between a measured pupil radius and a radial distance between the two boundaries.

6. An iris camera according to claim 1, wherein the control module is arranged to generate a pulsed control signal to control the light source to emit pulses of light.

7. An iris camera according to claim 6, wherein the control module is arranged to generate a control signal having pulses provided at random intervals to control the light source to emit pulses of light at random time intervals which are not predictable by the user.

8. An iris camera according to claim 6, wherein the control module is arranged to generate a control signal having pulses provided at time intervals corresponding to selected captured image frames of the iris wherein the selected image frames are not predictable by the user.

9. An iris camera according to claim 1, further comprising the light source.

10. An iris camera according to claim 9, wherein the light source is positioned within the iris camera to face the iris and directly in front of the iris being imaged.

11. An iris camera according to claim 9, wherein the light source is positioned within the iris camera below a horizontal level of the iris being imaged.

12. An iris camera according to claim 9, wherein the light source comprises light emitting diodes (LEDs).

13. An iris camera according to claim 12, wherein the LEDs are arranged to emit a broad spectrum of wavelengths making up a white light.

14. An iris camera according to claim 2, wherein the control module is arranged to generate a failure signal if, after a predetermined time period or a predetermined number of cycles of image re-capture and re-analysis, pupil dilation of the iris is still greater than the predetermined threshold.

15. A method of controlling the quality of iris captured images output from an iris camera, the method comprising:
obtaining an initial image of an iris of a user from the camera;
measuring pupil dilation of the iris using image analysis techniques on the captured image;
comparing the measured pupil dilation with a predetermined threshold;
illuminating the iris and capturing a further image, if the determined pupil dilation is greater than the threshold; and
outputting a validated iris image, if the comparing step shows that the determined pupil dilation is not greater than the predetermined threshold.

16. A method according to claim 15, wherein the measuring step comprises:
locating a pupil-iris boundary and an iris-sclera boundary in the captured image by searching for regions having a sudden change in contrast; and
calculating a measure of pupil dilation based on locations in the captured image of the boundaries with respect a location of a pupil centre.

17. A method according to claim 15, further comprising calculating the measure of pupil dilation to be a ratio in the captured image between a measured pupil radius and a radial distance between the two boundaries.

18. A non-transitory computer readable storage medium having instructions stored therein, the instructions executable by a processor and comprising:
obtaining an initial image of an iris of a user from the camera;
measuring pupil dilation of the iris using image analysis techniques on the captured image;
comparing the measured pupil dilation with a predetermined threshold;
illuminating the iris and capturing a further image, if the determined pupil dilation is greater than the threshold; and
outputting a validated iris image, if the comparing step shows that the determined pupil dilation is not greater than the predetermined threshold.

19. The computer readable storage medium of claim 18, wherein the instructions for measuring further comprise instructions for:
locating a pupil-iris boundary and an iris-sclera boundary in the captured image by searching for regions having a sudden change in contrast; and
calculating a measure of pupil dilation based on locations in the captured image of the boundaries with respect a location of a pupil centre.

20. The computer readable storage medium of claim 18, further comprising calculating the measure of pupil dilation to be a ratio in the captured image between a measured pupil radius and a radial distance between the two boundaries.
